(19) United States
(12) Patent Application Publication
(10) Pub. No.: US 2023/0260656 A1
(43) Pub. Date: Aug. 17, 2023

(54) COHORT STRATIFICATION INTO ENDOTYPES

(71) Applicant: BenevolentAI Technology Limited, London (GB)

(72) Inventors: Andrea MARTINEZ, London (GB); Antonios POULAKAKIS-DAKTYLIDIS, London (GB); Hamish TOMLINSON, London (GB); Pijika WATCHARAPICHAT, London (GB); Sera Aylin CAKIROGLU, London (GB)

(73) Assignee: BenevolentAI Technology Limited, London (GB)

(21) Appl. No.: 18/300,623

(22) Filed: Apr. 14, 2023

Related U.S. Application Data
(63) Continuation of application No. PCT/GB2021/052570, filed on Oct. 5, 2021.

(30) Foreign Application Priority Data
Oct. 16, 2020 (GB) 2016469.5

(51) Int. Cl. G16H 50/20; G16H 70/60; G16H 10/60

(52) U.S. Cl. CPC G16H 50/20; G16H 10/60; G16H 70/60

(57) ABSTRACT

A system for identifying a target for the treatment of a primary disease is provided. The system comprises: an input module configured to receive data for studying the primary disease, the data relating to individuals of a cohort; an encoder configured to use machine learning to encode the data as latent variables; an interpretation module configured to interpret the latent variables to stratify the individuals of the cohort into endotypes of the primary disease; and an identification module configured to identify a target that is associated with one of the endotypes.

### CROSS-REFERENCE TO RELATED APPLICATIONS

The present application is a bypass continuation of International Application No. PCT/GB2021/052570, filed on Oct. 5, 2021, which in turn claims priority to UK Application No. 2016469.5, filed on Oct. 16, 2020. Each of these applications is incorporated herein by reference in its entirety for all purposes.

### FIELD OF INVENTION

The present application relates to systems and methods for stratifying a cohort of individuals into disease endotypes. The presently disclosed techniques find particular application in the fields of translational medicine and drug discovery where there is a need to understand the various endotypes of a disease and develop treatments for them.

### BACKGROUND

In order to study a disease of interest, data relating to a cohort of individuals having the disease can be used to produce a model. Machine learning models can be used to stratify the cohort of individuals into subgroups that correspond to endotypes of the disease, which is useful in medicine and drug discovery because different disease endotypes are typically associated with different underlying biological mechanisms. If an endotype is well understood, a drug target that is relevant to the biological mechanism of that endotype can be identified for the development of a potential treatment. In order to make the best use of machine learning methods for developing treatments for diseases, it is important to know in detail what data to put into the machine learning model and how to interpret the results of the machine learning model.

Accordingly, there is a need for an improved technique of using machine learning methods to understand disease endotypes and develop corresponding treatments.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter.

In a first aspect, the present disclosure provides a computer-implemented method of identifying a target for the treatment of a primary disease, the method comprising: receiving data for studying the primary disease, the data relating to individuals of a cohort; using machine learning to encode the data as latent variables; interpreting the latent variables to stratify the individuals of the cohort into endotypes of the primary disease; and identifying a target that is associated with one of the endotypes.

Optionally, the data relate to biological or health-related features of the individuals. Optionally, the data relate to comorbid diseases associated with the individuals. Optionally, the data relate to physiological measurements, medications or biomarkers associated with the individuals. Optionally, the data relate to omics or genetic data associated with the individuals. Optionally, the data relate to longitudinal information about the individuals.

Optionally, the computer-implemented method comprises transforming the data 102 into a canonical format. Optionally, the computer-implemented method comprises obtaining electronic health record data relevant to the primary disease in a structure ready for machine learning.

Optionally, the machine learning comprises using a latent variable model such as a matrix or tensor factorisation algorithm to operate on: a first matrix representing a mapping of individuals to latent variables; and a second matrix representing a mapping of features of the individuals to latent variables. Optionally, the features of the individuals comprise diseases. Optionally, the machine learning comprises using an autoencoder or a variational autoencoder.

Optionally, interpreting the latent variables comprises performing enrichment analysis. Optionally, interpreting the latent variables comprises applying a sparsification technique. Optionally, the computer-implemented method comprises using the interpretation of the latent variables to identify endotypes of the primary disease.

Optionally, the computer-implemented method comprises interpreting the latent variables to identify one or more secondary diseases. Optionally, the computer-implemented method comprises identifying one or more of the latent variables that represent a particular secondary disease. Optionally, the computer-implemented method comprises generating a comorbidity enrichment table using a comorbidity classification system such as the Elixhauser comorbidity index. Optionally, interpreting the latent variables comprises computing association scores between diseases represented by the latent variables. Optionally, the computer-implemented method comprises identifying endotypes of the primary disease using comorbidities the latent variables represent.

Optionally, the computer-implemented method comprises interpreting the latent variables to identify characteristics of the individuals. Optionally, the computer-implemented method comprises associating the latent variables with targets such as genes, proteins or intermediate products such as RNA using omics or genetic data.

Optionally, one or more of the latent variables is associated with: the target, or an entity that is functionally related to the target via upstream or downstream regulation, one or more quantitative trait loci, or one or more other gene or protein interactions. Optionally, the target is associated with the primary disease and with a secondary disease.

Optionally, the computer-implemented method comprises using feedback from machine learning and/or from interpreting the latent variables to assist in ranking disease-specific machine learning model hyperparameters based on their performance.

In a second aspect, the present disclosure provides a computer-readable medium storing code that, when executed by a computer, causes the computer to perform any method provided by the present disclosure.

In a third aspect, the present disclosure provides a system for identifying a target for the treatment of a primary disease, the system comprising: an input module configured to receive data for studying the primary disease, the data relating to individuals of a cohort; an encoder configured to use machine learning to encode the data as latent variables; an interpretation module configured to interpret the latent variables to stratify the individuals of the cohort into endotypes of the primary disease; and a target identification module configured to identify a target that is associated with one of the endotypes.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1 is a block diagram of a system for stratifying a cohort of individuals to identify a target for the treatment of a disease according to an embodiment of the invention;

FIG. 2 is a flow chart of a method that may be carried out by the above system according to an embodiment of the invention;

FIG. 3 is a block diagram showing example input data that may be received by the above system according to an embodiment of the invention;

FIG. 4 is a block diagram showing example interpretation steps that may be carried out in accordance with the above method;

FIG. 5 is a flow chart showing a method of cohort stratification according to another embodiment of the invention;

FIG. 6 is a flow chart showing a method of cohort stratification according to a further embodiment of the invention;

FIG. 7 is a schematic diagram of an autoencoder suitable for use in embodiments of the invention; and

FIG. 8 is a block diagram of a computer hardware suitable for implementing embodiments of the invention.

Common reference numerals are used throughout the figures to indicate similar features.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

In accordance with the invention, data relating to a cohort of individuals is provided as inputs to a machine learning model. To learn about a disease, the cohort is selected to include individuals who have the disease of interest. For example, individuals may be selected on the basis of a disease or diagnosis code in a patient database. Alternatively, individuals may be selected on the basis of other indicators of the disease, such as physiological measurements or medications the individual is taking. These codes or indicators may be sourced from a patient or other suitable database.

When the cohort has been adequately defined, for example by way of using disease codes, a range of data is collected about the individuals of the cohort. These data are useful for studying the disease and are provided as part of the input for the machine learning model. The data that are collected represent characteristics or features of the individuals relating to their biology or health, and as such can be useful for separating a seemingly homogenous cohort of individuals who have a disease into subgroups that correspond to disease endotypes. ‘Disease endotypes’ or simply ‘endotypes’ are subtypes of a disease that have different underlying biological mechanisms.

The data that is collected about the individuals may for example include comorbidity data that indicate other diseases the individuals have in addition to the primary disease. The collected data may additionally or alternatively comprise clinical measurements relevant to the primary disease, age, gender and, if the data source comprises longitudinal data about the individuals, survival times. Further examples of the collected data include blood test results, physiology test results such as electrocardiograms (ECG) and spirometry test results, imaging results such as magnetic resonance imaging (MRI) results, survey results of relevant lifestyle factors such as diet and alcohol intake, family medical history, body composition, and medical history of the individuals including for example histories of medications and medical procedures. Examples of omics include transcriptomic or proteomic data derived from disease-relevant tissue samples of the individuals. Examples of genetic data include genotyping array data or whole genome sequencing of the individuals.

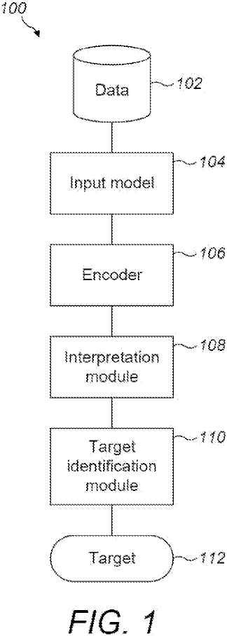

With reference to FIG. 1, a system 100 for stratifying a cohort of individuals in accordance with the invention comprises an input module 104 configured to receive data 102 for studying a primary disease. In this document, the term ‘primary disease’ refers to the disease of interest that is being studied through the use of machine learning. If there are comorbidities, then other diseases individuals of the cohort have in addition to the primary disease will be referred to as secondary diseases.

The system 100 further comprises an encoder 106 configured to use machine learning to encode the data 102 as latent variables. Latent variables are inferred variables that represent non-observable features hidden in the input data about the cohort of individuals. As a result, the latent variables may reveal groupings of biological and other features of the cohort that enable the model to separate out endotypes of the disease. Once the cohort has been stratified into latent variables that represent different disease endotypes, this opens the possibility of interrogating the latent variables to learn more about the underlying biological mechanisms of the endotypes. It will be appreciated that in some examples there may be a one-to-one relationship between latent variables and endotypes, while in other examples there could be an endotype represented by more than one latent variable.

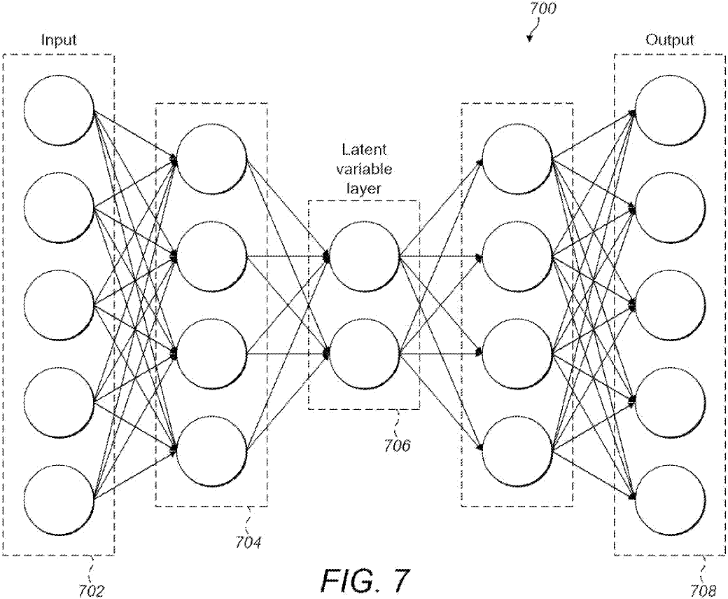

A variety of machine learning methods may be used to encode the data 102 as latent variables. For example, latent variable models such as matrix or tensor factorisation algorithms may be used to approximate a full data matrix as a product of two or three lower dimensional matrices, where one matrix represents mapping of individuals of the cohort to latent variables (the ‘latent matrix’) and the others represent mapping of features, such as diseases, represented along the other dimensions of the input matrix or tensor to latent variables (the ‘loading matrix’). Other suitable machine learning methods for generating latent variables include the use of an autoencoder or a variational autoencoder. The use of an autoencoder is described below in relation to FIG. 7.

When the data 102 has been encoded as latent variables, the latent variables are interpreted to identify those that are statistically significant and have a biological interpretation or meaning. For example, latent variables may be identified that are statistically significantly associated with selected features such as measures of disease progression. Latent variables that are statistically significant and have a biological meaning may represent endotypes of the primary disease and may as a result provide opportunities for developing new or repurposing known treatments for the disease. They may also provide opportunities for early detection or prevention of the primary disease as well as non-pharmaceutical interventions for treating the primary disease. As such, the system 100 comprises an interpretation module 108 configured to interpret the latent variables to stratify the individuals of the cohort into endotypes of the primary disease.

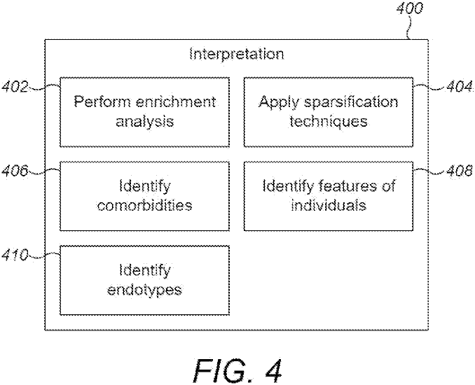

A range of interpretation techniques may be utilised to build up a biological characterisation of the latent variables. These techniques include enrichment analysis and sparsification techniques which enable statistically significant latent variables with biological meaning to be identified. These techniques and the way they are used according to the invention are described below in relation to FIG. 4.

Once the latent variables have been interpreted to stratify the cohort into endotypes, the endotypes are used to identify one or more potential drug targets. As such, the system 100 comprises a target identification module 110 configured to identify a target 112 that is associated with one of the endotypes. In typical examples, the target 112 comprises a complex biological molecule, or part of a complex biological molecule, such as a deoxyribonucleic acid (DNA), ribonucleic acid (RNA) or protein whose biological function can be regulated by a drug. For example, the target may comprise a gene that is relevant to the mechanism of a disease endotype and may be up or down regulated by a drug to provide a treatment for the disease.

In many examples, the target is associated with a latent variable that represents the disease endotype. In this case, a potential treatment for the disease could comprise a drug that modifies a gene associated with the latent variable. However, there may also be examples in which the target is not itself associated with the latent variable, but is an upstream regulator of an entity such as a gene or protein that is associated with the latent variable. In this case, a potential treatment for the disease could comprise a drug that modifies the upstream target and influences the underlying disease mechanism via downstream regulation. However, in some such cases it may be that a more effective treatment uses a target that is itself associated with the latent variable. In some examples, a latent variable may be associated with an entity that is functionally related to the target via upstream or downstream regulation, as found in a protein-protein interaction (PPI) network or colocalistion with quantitative trait loci.

The target identification module 110 may be configured to determine an association between a target 112 and a disease endotype using any suitable analytical method. For example, statistical tests of association between a latent variable that represents an endotype and omics data are performed to find one or more suitable targets for the endotype. Suitable statistical tests of association may include genome-wide association study (GWAS), differential expression or any bioinformatic workflow appropriate to the available data.

The statistical tests may be used to provide a probability that the latent variable is associated with the target, and a threshold probability may be applied to decide whether there is an association between the latent variable and the target. In some examples, the target identification module 110 may be configured to annotate a latent variable with targets that sufficiently regulate one or more of the entities such as genes or proteins associated with the latent variable. In these or other examples, the target identification module 110 may be configured to identify targets that are relevant to a disease mechanism of an endotype identified by the interpretation module 108.

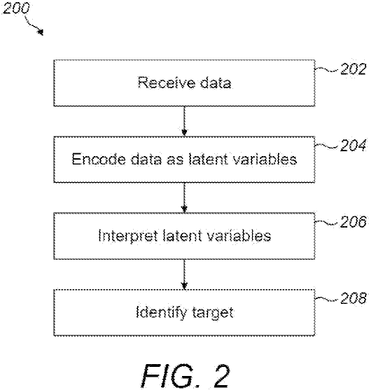

With reference to FIG. 2, the present disclosure extends to a computer-implemented method 200 of identifying a target for the treatment of a primary disease. The method 200 may be carried out by the system 100 of FIG. 1 and comprises: receiving 202 data for studying the primary disease, the data relating to individuals of a cohort; using machine learning to encode 204 the data as latent variables; interpreting 206 the latent variables to stratify the individuals of the cohort into endotypes of the primary disease; and identifying 208 a target that is associated with one of the endotypes.

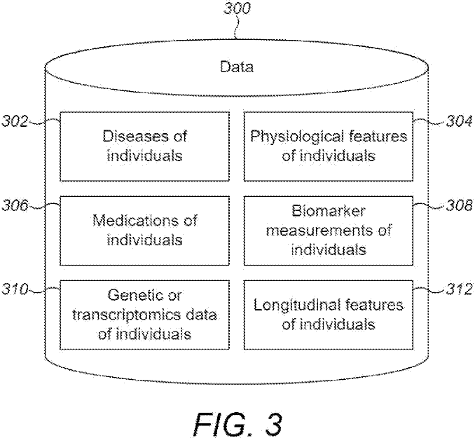

Referring to FIG. 3, an exemplary set of data 300 provides an example of the data 102 that is received by the system 100 according to an embodiment of the invention. The data 102 relate to individuals of a cohort that is relevant for studying a disease of interest and may be obtained from a range of data sources. Suitably, the data 102 relate to biological or health-related features of the individuals of the cohort and are useful for studying the disease. A suitable data set 102 may, for example, be based on data from approximately 100 individuals, although this figure is non-limiting and not intended as a guide.

Non-limiting examples of suitable data 102 may comprise data relating to comorbid diseases associated with the individuals. In this case, the data 102 may comprise disease codes or diagnosis codes or other suitable representations of diseases that indicate comorbid diseases an individual has along with the primary disease. The data 102 may additionally or alternatively comprise data that relates to physiological measurements, medications or biomarkers associated with the individuals. These data may comprise clinical data or other suitable patient or customer data which may be based on biomedical measurements that have been made on the individuals or on survey data relating to other suitable social or lifestyle factors that may be relevant. Biomarkers are indicators of a particular disease state or other physiological state that may be useful for studying the primary disease. For example, the biomarker may relate to the severity of the primary disease or to the presence of a secondary comorbid disease. The data 102 may additionally or alternatively comprise data relating to omics data associated with the individuals such as genetic data, transcriptomics data or data relating to the presence of particular proteins. In this case, the data 102 may for example comprise gene expression data, presence of a gene or gene variant, genotyping data, methylation data or copy number variation data. Finally, the data 102 may comprise longitudinal information about the individuals, for example resulting from a longitudinal study. Longitudinal information may for example relate to the age of individuals at disease onset and at key stages of disease progression, age at onset of comorbidities and the nature of the comorbidities, and survival times.

In the example of FIG. 3, the exemplary data set 300 comprises diseases of individuals (indicating comorbidities) 302, physiological features of individuals 304, medications being taken by individuals 306, biomarker measurements exhibited by individuals 308, genetic or transcriptomics data of individuals 310 and longitudinal features of individuals 312. It will be appreciated that other exemplary data sets may comprise other molecular data of individuals in addition to or alternatively to the genetic or transcriptomics data.

The data 102 may be obtained from various sources. For example, the data 102 may comprise electronic health records data obtained from a health or medical database such as the UK Biobank. The data 102 may additionally or alternatively comprise data from non-clinical data services that relate to individuals’ health and biology such as data from the personal genomics and biotechnology company 23andMe. For survival times, the data 102 may additionally or alternatively comprise data from a death registry. For disease-related information, the data 102 may additionally or alternatively comprise data from a disease registry.

Any missing data can be handled by adopting a probabilistic approach in which the model that encodes the data as latent variables treats missing data as unknown parameters that can be statistically inferred.

Some or all of the data 102 may need to be transformed into a canonical format in order for machine learning models to be applied to the data 102. In embodiments, the system 100 is configured to transform the data 102 into a canonical format. In some embodiments, some or all of the data 102 may be obtained in a structure ready for machine learning modelling. In this case, the system 100 is configured to receive data 102 in a structure ready for machine learning modelling. For example, the system 100 may be configured to obtain electronic health record data relevant to the primary disease in a structure ready for machine learning.

Once the data 102 has been encoded 204 as latent variables, the latent variables are interpreted 206 to identify statistically significant latent variables that have biological meaning. Optionally, the interpretation 206 may be used to identify latent variables that have a clinical meaning. Typically, hundreds or thousands of latent variables require interpretation by model introspection.

Various interpretation techniques may be used. For example, enrichment analysis may be performed on latent variables to determine features such as genes or other characteristics of individuals they represent. Enrichment analysis refers to a statistical analysis, for example using a Fisher’s Exact Test, to identify over or under-representation of particular features by a latent variable. Latent variables may also be interpreted to determine relevant clinical measurements, age, gender and other characteristics of individuals they are enriched for. An endotype may be associated with one or more of the latent variables on the basis of the results of enrichment analysis.

Enrichment analysis may additionally or alternatively be used to determine comorbidities latent variables are enriched for. This approach is used to find secondary diseases encoded by the latent variables that cooccur with the primary disease. If a particular secondary disease is associated with a latent variable, then the latent variable may represent an endotype of the primary disease. In this case, it may be possible to find a target that is associated with both the primary disease and the secondary disease. In this case, the target may provide a viable treatment for both diseases. Alternatively, the target may provide a treatment for the primary disease that is particularly well suited for the cohort subgroup represented by the latent variable. In this case, the treatment may be well suited for the subgroup by virtue of being more effective for that subgroup than other available treatments or by virtue of having fewer side effects for that subgroup than other available treatments.

Some latent variables may be associated with a set of secondary diseases, thereby representing a comorbidity cluster. Such clusters can represent an underlying clinical process - i.e. a disease endotype.

As a result, it may be suitable to generate a comorbidity enrichment table for latent variables that represent comorbidity clusters. The comorbidity enrichment table contains enrichment analysis results that indicate the comorbid diseases encoded by the latent variables. A suitable example is a table which contains enrichment analysis results based on the Elixhauser Comorbidity Index which is a method of categorising comorbidities of patients in common disease themes based on the International Classification of Diseases diagnosis codes. A similar comorbidity enrichment table may be generated using other suitable comorbidity indexes based on a disease theme the user is interested in. The aim is to identify what disease theme an endotype is enriched for. A disease theme refers to a set of comorbidities that are known to arise in combination with a primary condition. For example, if diabetes is the primary disease, then a disease theme of ‘complicated’ or ‘advanced’ could combine diabetes with its known follow-up complications such as retinopathy and kidney disease.

Comorbidity clusters may additionally or alternatively be characterised by generating association scores among diseases. For example, it would be suitable to define an association score such that if two diseases have a high association score, this means that they frequently cooccur. A diagram representing a disease-disease network may be generated such that if two diseases are associated (for example because they frequently cooccur), then they are connected by an edge.

It may be suitable to characterise latent variables from the point of view of characteristics of the individuals other than associated disease codes and diagnosis codes. For example, characteristics of individuals such as clinical measurements, age, gender and survival rates that show a divergence from a control group may be used to rank latent variables to highlight those that represent interesting subgroups of the cohort of individuals. An individuals’ characteristics table may be generated that contains enrichment analysis results indicating the characteristics of individuals associated with the latent variables. Once latent variables have been associated with endotypes, characteristics of individuals that are typical for each endotype may be determined. In some examples, the characteristics of the individuals encoded by the latent variables may be used to identify the endotypes represented by the latent variables. An aetiology table may additionally or alternatively be generated that indicates aetiologies (i.e. disease causes) associated with the latent variables. Additionally or alternatively to the enrichment analysis, suitable statistical methods may be used to determine statistical associations that the latent variables have with disease progress, survival times, and other relevant biological or clinical parameters.

Sparsification strategies may be applied to aid the interpretation of the latent variables. For example, suitable sparsification strategies may be applied to assign individuals and disease codes to an endotype. These sparsification strategies may be implicit in the model architecture or applied as post-processing. If sparsification is applied as post-processing, a threshold may be dynamically found based on the distribution of values in the latent variable according to criteria such as place in a cumulative distribution function of the latent values or a probability distribution function of the latent.

On the basis of the results of the interpretation techniques, biological and optionally clinical characterisations of the latent variables are generated. This enables identification of endotypes that are represented by the latent variables.

Referring to FIG. 4, an exemplary interpretation step 400 comprises performing enrichment analysis 402, applying sparsification techniques 404, identifying comorbidities 406, identifying features of individuals 408 and identifying endotypes 410.

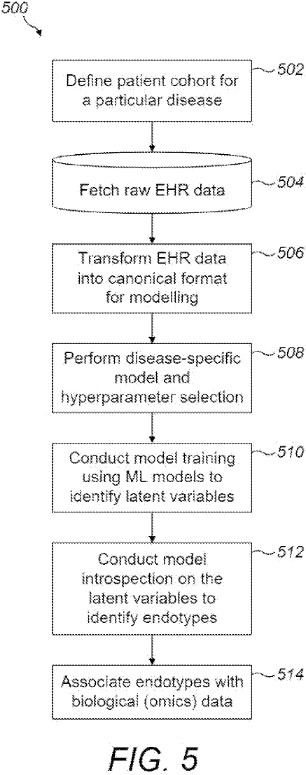

FIG. 5 shows a method 500 of stratifying a cohort of individuals to identify a target for the treatment of a disease according to an embodiment of the invention. The method 500 comprises defining 502 a patient cohort for a particular disease of interest. For example, the patient cohort may be defined as all patients from a particular data source of patient records that are associated with a particular disease code or diagnosis code. Other suitable methods for extracting data relating to patients having the disease of interest may additionally or alternatively be used. In this embodiment, the data comprises electronic health record (EHR) data.

The method 500 comprises fetching 504 raw EHR data from a suitable data source such as the UK Biobank. To obtain the raw EHR data, a programming language such as Python may be used to specify a set of rules for extracting certain patient data relating to the defined cohort.

The method then comprises transforming 506 the EHR data into a canonical format suitable for machine learning models to be applied to the data.

A suitable model is selected 508 for the disease of interest from a set of eligible machine learning methods, along with its optimal hyperparameters for that disease. Examples of eligible machine learning methods may include matrix factorisation algorithms or the use of an autoencoder or a variational autoencoder.

Once the model and hyperparameters have been selected, the machine learning model is trained 510 to identify latent variables from the selected EHR data. The latent variables represent features of the inputted EHR data and enable the model to separate out endotypes of the disease of interest. For example, the latent variables may represent groupings of biological or clinical features of the patient cohort that together may represent an underlying biological mechanism of the disease. By representing an underlying disease mechanism, a latent variable may be associated with an endotype of the disease. In this way, the latent variables may be used to stratify the patients of the cohort into endotypes according to different biological mechanisms of the same disease.

Some disease endotypes are associated with one or more particular secondary diseases, forming a comorbidity cluster with the primary disease (i.e. the disease of interest). In this case, the comorbidity cluster represented by a latent variable can be used to determine the endotype the latent variable represents. If the comorbidity clusters represented by the latent variables can be identified, this can assist in the stratification of the patient cohort into endotypes.

In order to interpret the latent variables and identify endotypes, model introspection 512 is carried out on the latent variables. Model introspection 512 can be used to interpret hundreds or thousands of latent variables using techniques such as enrichment analysis and methods for identifying statistical associations with variables of interest. For example, such techniques can be used to determine the features such as disease codes and patient characteristics that the latent variables represent. By interpreting the latent variables, model introspection 512 can be used to build up a biological or clinical characterisation of the latent variables. For example, it may be determined that a latent variable represents the over-representation of a particular set of comorbidities or a patient characteristic such as a gender identity or a disease risk factor. The characterisation of a latent variable is used to identify an underlying biological disease mechanism that is represented by the latent variable and to associate the latent variable with an endotype of the primary disease.

The latent variables may be annotated with outputs of the model introspection step. For example, clinical and statistical metadata relating to clinical and biological interpretations of the latent variables and their level of statistical significance may be used to annotate the latent variables as part of the characterisation of the latent variables.

The outputs of model introspection may additionally or alternatively be presented to the user in the form of graphical representations of summary statistics and other representations of the interpretation of the latent variables. These may take the form of heat maps or density plots, tables, graphs and so on. In an example, a table of over-represented comorbidities defined for example by the Elixhauser classification system may be presented graphically to the user to show disease themes the latent variables are enriched for. Comorbidities represented by latent variables may alternatively or additionally be represented by a comorbidity diagram or map in which two diseases in the map are connected if they occur together frequently. An aetiology table may be provided graphically summarising common disease causes across latent variables or patient subgroups.

Endotype reports may be generated to be graphically presented to the user depending on the user’s interest in particular introspection findings. For example, comorbidities relevant to an endotype (including their sequential occurrence in the progression of the primary disease if relevant) may be presented in the form of a heat map plot. In a second example, a bar chart may be generated to show the importance weights the model assigns to the most relevant disease codes in an endotype. In a third example, a density plot may use disease categories to depict the cooccurrence of different physiological systems relevant to an endotype in the form of a density plot. In a fourth example, a patient characteristics group plot may be used to show both general and disease-specific characteristics of the patient subgroup, including distributions of age if time-dependency for a disease was taken into account in the inputted EHR data and model. In a fifth example, a pairwise plot of clinical covariates may show summary statistics between the patients associated with the endotype and patients outside this subgroup. The endotype reports may be collated and written in a markup format such as hyper text markup language (HTML) so that the user can view the reports from a browser and use links to navigate between the findings.

Following model introspection, the identified endotypes are associated 514 with omics or genetic data to identify a target for treating the primary disease. For example, the genetic data may comprise genotyping data which may be analysed using GWAS or other statistical or computational methods for associating genetic variations with case-control data or quantitative phenotypes derived from endotypes, for example.

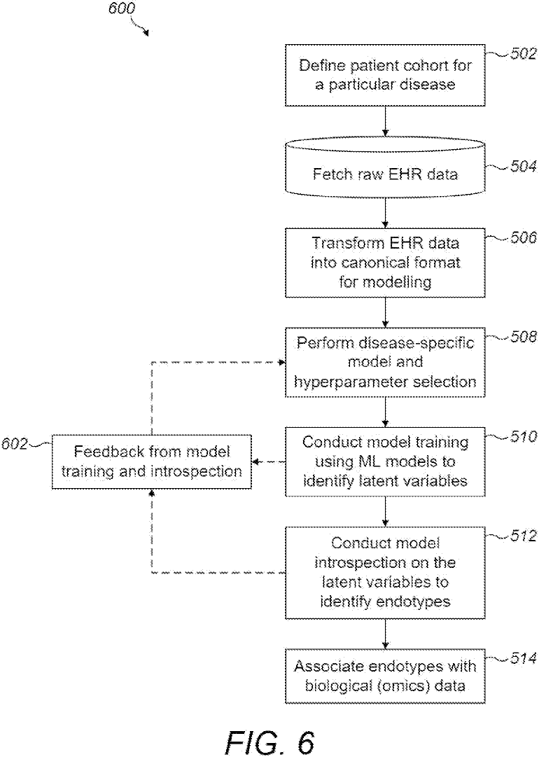

Referring to FIG. 6, an embodiment of the invention comprises using feedback 602 to assist in the assessment of disease-specific model hyperparameters. The feedback is obtained from the machine learning step of training the model and from the interpretation step of model introspection. The feedback is used to assess the hyperparameters based on their performance, and optionally to rank the hyperparameters in cases where direct comparison is suitable. It will be appreciated that some diseases have higher numbers of comorbidities and/or larger cohorts, for example as a result of a higher disease prevalence. Consequently, different numbers of latent variables may be needed to capture the latent factors of variation in the data. Similarly, different model parameters such as the number of iterations may be tuned to converge to the ideal representations, and this can differ depending on the number of latent variables and the cohort size. Furthermore, some diseases vary greatly over time with respect to onset and comorbidities, and so applying time-specific transformations may be appropriate. Thus, it is suitable to review the introspection results after a first proposal of default parameter settings, and to use the outputs to drive changes to the machine learning model settings.

The steps 204 and 510 of encoding data as latent variables in methods 200 and 500 above (and shown in FIGS. 2 and 5 respectively) may be achieved using a range of techniques, including supervised and unsupervised machine learning methods.

In an example approach, a matrix or tensor (a higher-dimensional generalization of matrices) factorisation technique is used. Matrix or tensor factorisation techniques operate on the basis of decomposing a full data matrix or tensor that is inputted into a machine learning model into two or more lower dimensional matrices. According to this approach, data 102 may be simplified into a first matrix that maps individuals of a cohort to latent variables (the ‘latent matrix’) and a second matrix that maps features such as diseases to latent variables (the ‘loading matrix’). In the case of a tensor factorisation, a third matrix may map the third dimension of the input tensor (for example age at disease diagnosis) to latent variables, and so on. In other examples, the second matrix may map other features such as medications, medical procedures and physiological parameters to latent variables.

In another example approach, an autoencoder is used. Referring to FIG. 7, an autoencoder 700 may be used to encode latent variables from the data. In this example, an input vector 702 is passed through a neural network of one or more layers of hidden nodes 704 to an intermediate layer with fewer nodes than the input - that is with a dimensionality reduction 706. These nodes are connected to additional nodes in additional layers to a series of output nodes 708 of the same dimensionality as the input layer. Such a system may be trained to reconstruct input data at the output, resulting in compact, lower dimensional representations of different inputs in the intermediate latent variable layer 706.

As an alternative to this approach, a variational autoencoder may be used that additionally encodes a standard deviation vector, which is sampled at the latent variable stage before being decoded back to the original input.

A further example approach that may be used additionally or alternatively comprises the use of unsupervised machine learning techniques or other clustering algorithms, such as k-means, mixture models, density-based spatial clustering of applications with noise (DBSCAN), or other suitable methods. These methods may be linear or non-linear. It will be appreciated that latent variables may be generated using one of the above methods or a combination of those methods.

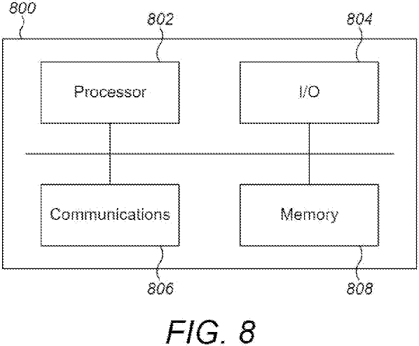

A computer apparatus 800 suitable for implementing methods according to the present invention is shown in FIG. 8. The apparatus 800 comprises a processor 802, an input-output device 804, a communications portal 806 and computer memory 808. The memory 808 may store code that, when executed by the processor 802, causes the apparatus 800 to perform the method 200 shown in FIG. 2.

In the embodiment described above the server may comprise a single server or network of servers. In some examples the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-Programmable Gate Arrays (FPGAs), Program-Specific Integrated Circuits (ASICs), Program-Specific Standard Products (ASSPs), System-On-a-Chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to “an” item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method of identifying a target for treatment of a primary disease, the computer-implemented method comprising:
receiving data for studying the primary disease, the data relating to individuals of a cohort;
using machine learning to encode the data as latent variables;
interpreting the latent variables to stratify the individuals of the cohort into endotypes of the primary disease; and
identifying a target that is associated with one of the endotypes.

2. The computer-implemented method of claim 1, wherein the data relate to biological or health-related features of the individuals.

3. The computer-implemented method of claim 1, wherein the data relate to comorbid diseases associated with the individuals.

4. The computer-implemented method of claim 1, wherein the data relate to physiological measurements, medications or biomarkers associated with the individuals.

5. The computer-implemented method of claim 1, wherein the data relate to one or more of: omics data associated with the individuals, genetic data associated with the individuals and longitudinal information about the individuals.

6. The computer-implemented method of claim 1, comprising transforming the data into a canonical format.

7. The computer-implemented method of claim 1, comprising obtaining electronic health record data relevant to the primary disease in a structure ready for machine learning.

8. The computer-implemented method of claim 1, wherein the machine learning comprises using a latent variable model such as a matrix or tensor factorisation algorithm to operate on:
a first matrix representing a mapping of individuals to latent variables; and
a second matrix representing a mapping of features of the individuals to latent variables; wherein the features of the individuals comprise diseases.

9. The computer-implemented method of claim 1, wherein the machine learning comprises using an autoencoder or a variational autoencoder.

10. The computer-implemented method of claim 1, wherein interpreting the latent variables comprises one or both of:
performing enrichment analysis; and
applying a sparsification technique.

11. The computer-implemented method of claim 1, comprising using the interpretation of the latent variables to identify endotypes of the primary disease.

12. The computer-implemented method of claim 1, comprising interpreting the latent variables to identify one or more secondary diseases and identifying one or more of the latent variables that represent a particular secondary disease.

13. The computer-implemented method of claim 12, comprising generating a comorbidity enrichment table using a comorbidity classification system.

14. The computer-implemented method of claim 12, wherein interpreting the latent variables comprises computing association scores between diseases represented by the latent variables.

15. The computer-implemented method of claim 12, comprising identifying endotypes of the primary disease using comorbidities the latent variables represent.

16. The computer-implemented method of claim 1, comprising associating the latent variables with targets such as genes, proteins or intermediate products such as RNA using omics or genetic data.

17. The computer-implemented method of claim 1, wherein one or more of the latent variables is associated with:
the target, or
an entity that is functionally related to the target via upstream or downstream regulation, one or more quantitative trait loci, or one or more other gene or protein interactions.

18. The computer-implemented method of claim 1, wherein the target is associated with the primary disease and with a secondary disease.

19. The computer-implemented method of claim 1, comprising using feedback from machine learning and/or from interpreting the latent variables to assist in ranking disease-specific machine learning model hyperparameters based on their performance.

20. A system for identifying a target for treatment of a primary disease, the system comprising:
an input module configured to receive data for studying the primary disease, the data relating to individuals of a cohort;
an encoder configured to use machine learning to encode the data as latent variables;
an interpretation module configured to interpret the latent variables to stratify the individuals of the cohort into endotypes of the primary disease; and
a target identification module configured to identify a target that is associated with one of the endotypes.
